(19) United States
(12) Patent Application Publication
(10) Pub. No.: US 2018/0070917 A1
(43) Pub. Date: Mar. 15, 2018

(54) INGESTIBLE ULTRASOUND DEVICE, SYSTEM AND IMAGING METHOD

(71) Applicant: Butterfly Network, Inc., Guilford, CT (US)

(72) Inventors: Jonathan M. Rothberg, Guilford, CT (US); Christopher Thomas McNulty, Guilford, CT (US); Nevada J. Sanchez, Guilford, CT (US); Tyler S. Ralston, Clinton, CT (US)

(73) Assignee: Butterfly Network, Inc., Guilford, CT (US)

(21) Appl. No.: 15/263,868

(22) Filed: Sep. 13, 2016

(51) Int. Cl. A61B 8/12; A61B 8/00; A61B 8/08; A61N 7/02

(52) U.S. Cl. CPC A61B 8/12; A61B 8/4472; A61N 7/022; A61B 8/4245; A61B 8/5207; A61B 8/4477

(57) ABSTRACT

An ingestible ultrasound device includes an electronic circuit assembly, including a plurality of ultrasonic transducers and control circuitry configured to control the plurality of ultrasonic transducers to generate and/or detect ultrasound signals; and an encapsulating medium that encapsulates the electronic circuit assembly.

### BACKGROUND

The present disclosure relates generally to ultrasound imaging. In particular, the present disclosure relates to an encapsulated, ingestible ultrasound device for internal use.

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a probe), sound waves are reflected off the tissue with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

In one embodiment, an ultrasound device includes an electronic circuit assembly, including a plurality of ultrasonic transducers and control circuitry configured to control the plurality of ultrasonic transducers to generate and/or detect ultrasound signals; and an ingestible encapsulating medium that encapsulates the electronic circuit assembly.

In another embodiment, an ultrasound device includes an electronic circuit assembly, including a plurality of ultrasonic transducers and control circuitry configured to control the plurality of ultrasonic transducers to generate and/or detect ultrasound signals; wherein at least a portion of the electronic circuit assembly is integrated on a same substrate with at least one ultrasonic transducer of the plurality of ultrasonic transducers; and an ingestible encapsulating medium that encapsulates the electronic circuit assembly.

In another embodiment, an ultrasound device includes an electronic circuit assembly, including a plurality of ultrasonic transducers and control circuitry configured to control the plurality of ultrasonic transducers to generate and/or detect ultrasound signals, wherein the control circuitry includes a timing and control circuit, a transmit circuit, and a receive circuit, the timing and control circuit configured to synchronize and coordinate the operation of the transmit circuit and the receive circuit; and an ingestible encapsulating medium that encapsulates the electronic circuit assembly.

In another embodiment, an ultrasound device includes an electronic circuit assembly, including a plurality of ultrasonic transducers and control circuitry configured to control the plurality of ultrasonic transducers to generate and/or detect ultrasound signals, wherein the control circuitry includes a memory device configured to store and/or buffer ultrasound image data therein; and an ingestible encapsulating medium that encapsulates the electronic circuit assembly.

In another embodiment, an ultrasound device includes an electronic circuit assembly, including a plurality of ultrasonic transducers and control circuitry configured to control the plurality of ultrasonic transducers to generate and/or detect ultrasound signals, wherein the control circuitry includes a power management circuit configured to manage power consumption within the device; and an ingestible encapsulating medium that encapsulates the electronic circuit assembly.

In another embodiment, a method of performing ultrasound imaging includes receiving, at a host device, ultrasound image data transmitted by an ultrasound device disposed internally within a subject, the host device located externally with respect to the subject, the ultrasound device including an electronic circuit assembly, having a plurality of ultrasonic transducers and control circuitry configured to control the plurality of ultrasonic transducers to generate and/or detect ultrasound signals, and an ingestible encapsulating medium that encapsulates the electronic circuit assembly.

### BRIEF DESCRIPTION OF THE DRAWINGS

Various aspects and embodiments of the disclosed technology will be described with reference to the following Figures. It should be appreciated that the figures are not necessarily drawn to scale. Items appearing in multiple figures are indicated by the same reference number in all the figures in which they appear.

FIG. 1 is a schematic diagram of an ultrasound imaging system including an ingestible ultrasound imaging device that is configured to wirelessly transmit ultrasound image data taken from a patient to a host device, in accordance with an exemplary embodiment;

FIG. 2 is a perspective view of one embodiment of the ingestible ultrasound imaging device of FIG. 1;

FIG. 3 is a sectional view of the ingestible ultrasound imaging device of FIG. 2;

FIG. 4 is another perspective view of an embodiment of the ingestible ultrasound imaging device of FIG. 1;

FIG. 5 is an exploded isometric view of the ingestible ultrasound imaging device of FIG. 4;

FIG. 6 is a perspective view of the electronic circuit assembly of the ingestible ultrasound imaging device of FIG. 4 and FIG. 5, shown in a folded configuration;

FIG. 7 is a perspective view of the electronic circuit assembly of FIG. 6 in an unfolded configuration;

FIG. 8 is a perspective view of a portion of the electronic circuit assembly of FIG. 7, according to an alternative embodiment;

FIG. 9 is a perspective view of a portion of the electronic circuit assembly of FIG. 7, according to another alternative embodiment;

FIG. 10 is a schematic cross-sectional view of the electronic circuit assembly of FIG. 6, illustrating a field of view of the ultrasonic transducer arrays;

FIG. 11 is a perspective view of a single transducer array chip embodiment of the electronic circuit assembly;

FIG. 12 is a perspective view of the electronic circuit assembly of FIG. 11 is a folded arrangement;

FIG. 13 is a schematic block diagram illustrating at least part of the functionality of the electronic circuit assembly;

FIG. 14 is a block diagram illustrating some of the electronic circuit assembly components in FIG. 13 in further detail;

FIG. 15 shows an illustrative arrangement of transducer cells of an ultrasonic transducer array in accordance with an exemplary embodiment; and

FIG. 16 shows an illustrative arrangement of transducer cells of an ultrasonic transducer array in accordance with another exemplary embodiment.

### DETAILED DESCRIPTION

A number of cancers are treatable if detected at an early stage, however the lack of reliable screening procedures results in their being undetected and untreated. For example, the impact of neoplastic disease (cancer) of the gastrointestinal (GI) tract is severe. In addition, there are other GI tract disorders that also require reliable screening and diagnostic procedures for early detection and treatment. Such disorders include, for example, irritable bowel syndrome, fluxional diarrhea, ulcerative colitis, collagenous colitis, microscopic colitis, lymphocytic colitis, inflammatory bowel disease, Crohn's disease, infectious diarrhea, ulcerative bowel disease, lactase deficiency, infectious diarrhea, amebiasis, and giardiasis.

Optical instruments such as endoscopes and colonoscopes may be inserted into upper and lower portions, respectively, of the GI tract but do not necessarily provide complete coverage since these instruments do not reach, for example, the jejunum and ileum portions of the small intestine. Even with devices that can optically scan the entire GI tract (such as by capsule endoscopy), only those conditions visible at the innermost layer (epithelium) of the tract are directly observable. Optical instruments are unable to determine conditions “at depth” (e.g., present within outer structures of the gut wall, such as muscle, connective tissue, lymphatic tissue, veins, arteries, and the like).

Accordingly, embodiments of the present disclosure provide an encapsulated, ingestible ultrasound device, system and method for patient imaging. Exemplary embodiments of the ingestible ultrasound device described herein may travel in the gastrointestinal (GI) tract to facilitate diagnosis of ailments of the GI tract, including those conditions located at innermost, intermediate and outermost layers of the GI tract.

Embodiments of the present disclosure are described more fully hereinafter with reference to the accompanying drawings, in which some, but not all, embodiments of the present disclosure are shown. Indeed, the present disclosure can be embodied in many different forms and should not be construed as limited to the embodiments set forth herein. Rather, these embodiments are provided so that this disclosure clearly satisfies applicable legal requirements. Like numbers refer to like elements throughout. As used herein, the terms “approximately”, “substantially,” and “about” may be used to mean within ±20% of a target value in some embodiments.

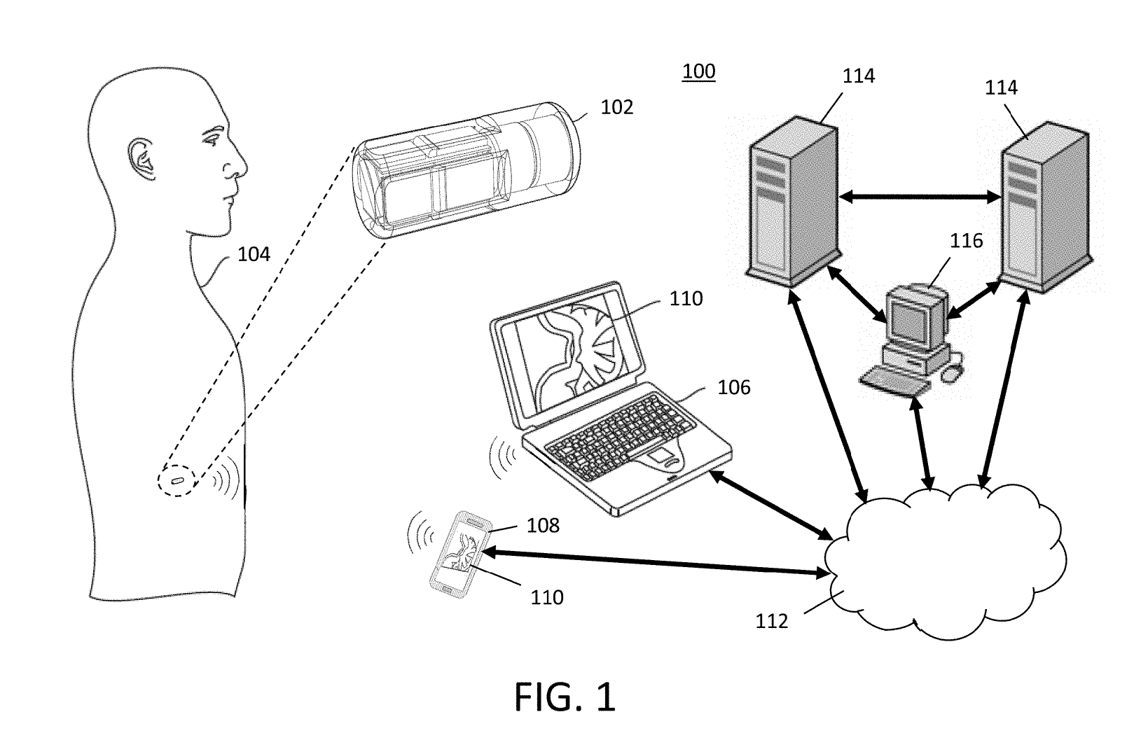

Referring initially to FIG. 1, there is shown a schematic diagram of an ultrasound imaging system 100 including an ultrasound imaging device 102 that is configured to be ingestible by a patient 104 and to wirelessly transmit ultrasound image data taken from the patient 104 to a host device such as, for example, a computer 106 or a mobile phone 108. Other host devices, however, are also contemplated (e.g., tablet device, desktop computer, etc.). Data wirelessly transmitted by the ultrasound imaging device 102 may be displayed as an ultrasound image 110 on a display screen of the host device (e.g., computer 106, mobile phone 108). In addition, any of the host devices 106, 108 may be communicatively coupled via a network 112 (e.g., local area network (LAN), wide area network (WAN), Internet, etc.) to any number of remote computing devices, such as a server(s) 114 or other personal workstation/computer 116. Such remote computing devices may be used, for example, to access and store ultrasound image data taken from the patient 104 for purposes including, but not limited to, remote diagnosis or telemedicine, and deep learning applications utilizing stored image data.

The ingestible ultrasound imaging device 102 may, in certain embodiments, be taken internally by the patient 104 by being swallowed in a pill form, for example. As the pill travels through the patient 104, the imaging device 102 may image the patient 104 and wirelessly transmit obtained data to one or more external host devices for processing the data received from the pill and generating one or more images of the patient 104. In other embodiments, it is contemplated that the ingestible ultrasound imaging device 102 may be administered internally to the patient 104 in a suppository form. In any case, an outer material of the ingestible ultrasound imaging device 102 may be formed from an inert, biocompatible material that is also acoustically conductive. In addition, in some embodiments, it is contemplated that the ingestible ultrasound imaging device 102 may be disposed of naturally by the patient 104 or manually retrieved and thereafter discarded. Alternatively, the ingestible ultrasound imaging device 102 may be retrieved for subsequent reuse, after cleaning and sterilizing. In embodiments where the ingestible ultrasound imaging device 102 is retrieved, the imaging data may be buffered and/or stored in memory within the device 102 for subsequent access and display. This feature may also be useful as a back-up for real time imaging in the event communication between the device 102 and the host device(s) is interrupted or disconnected.

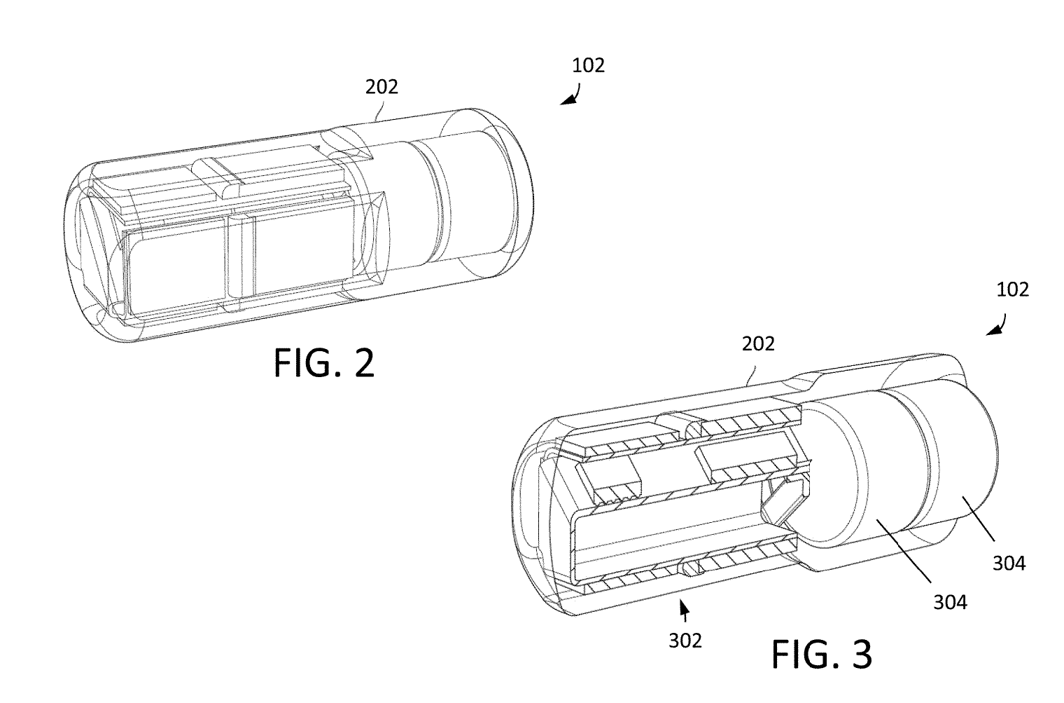

Referring now to FIG. 2 and FIG. 3, an embodiment of the ingestible ultrasound imaging device 102 is shown in further detail. An outer encapsulating medium 202 encapsulates an electronic circuit assembly 302 including one or more batteries 304 as particularly seen in the sectional view of FIG. 3. The encapsulating medium 202 may, in one embodiment, be optically transparent as depicted in the figures. Alternatively, the encapsulating medium 202 may include an opaque material. In the particular embodiment depicted in FIG. 2 and FIG. 3, the electronic circuit assembly 302 (and batteries 304) is potted in an acoustically conductive mold to define the outer encapsulating medium 202. One exemplary suitable material in this regard is Sylgard™, a silicone based encapsulant material available from Dow Corning. It will be appreciated that other potting materials may also be utilized, however. In addition, the encapsulating medium 202 may be selected and/or dimensioned so as to provide an acoustic lens effect with respect to acoustic energy transmitted by the device 102.

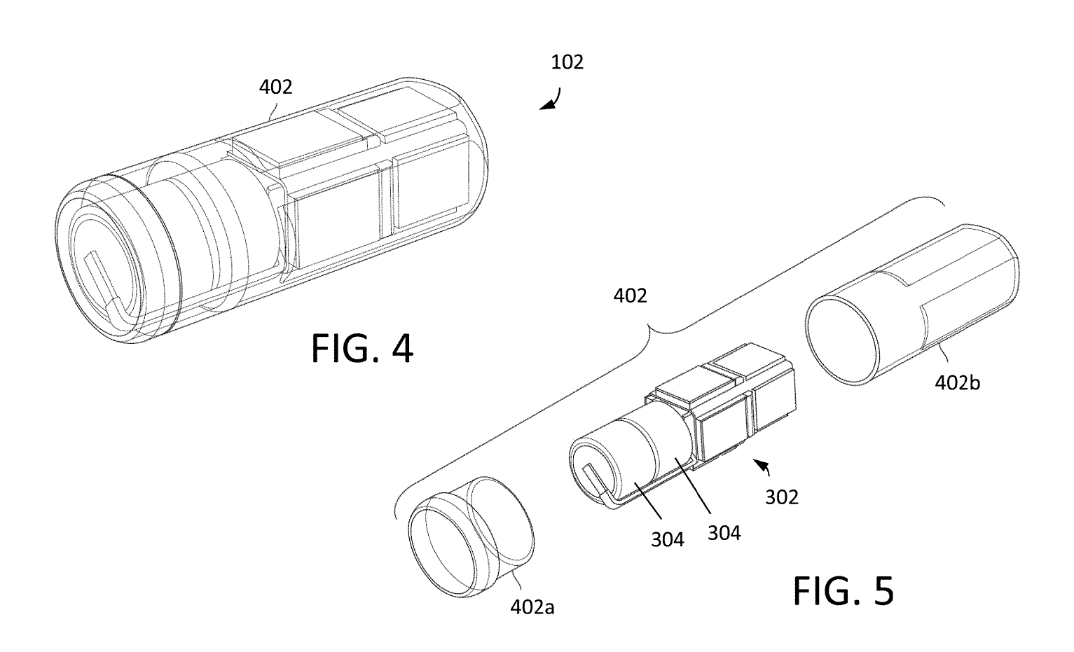

FIG. 4 and FIG. 5 illustrate an alternative embodiment of the ingestible ultrasound imaging device 102. In this embodiment, the electronic circuit assembly 302 is removably inserted into a two piece capsule 402 that serves as the encapsulating medium. In particular, FIG. 5 is an exploded isometric view of the ingestible ultrasound imaging device 102 showing the two piece capsule 402 as having outer housing portions 402a and 402b used to encase the electronic circuit assembly 302. As the electronic circuit assembly 302 is removably inserted into a two piece capsule 402, some clearance space may exist between portions of the electronic circuit assembly 302 and inner walls of the capsule 402. Thus, an acoustic coupling medium (not shown), such as an ultrasonic gel for example, may be introduced into one or both of the outer housing portions 402a, 402b prior to sealing. The ingestible ultrasound imaging device 102, whether in the form of the outer encapsulating medium 202 or the capsule may have dimensions suitable for oral or suppository administration. For example, the device 102 may have a length of about 25 millimeters (mm) and a diameter of about 11 mm. However, other device dimensions are also contemplated.

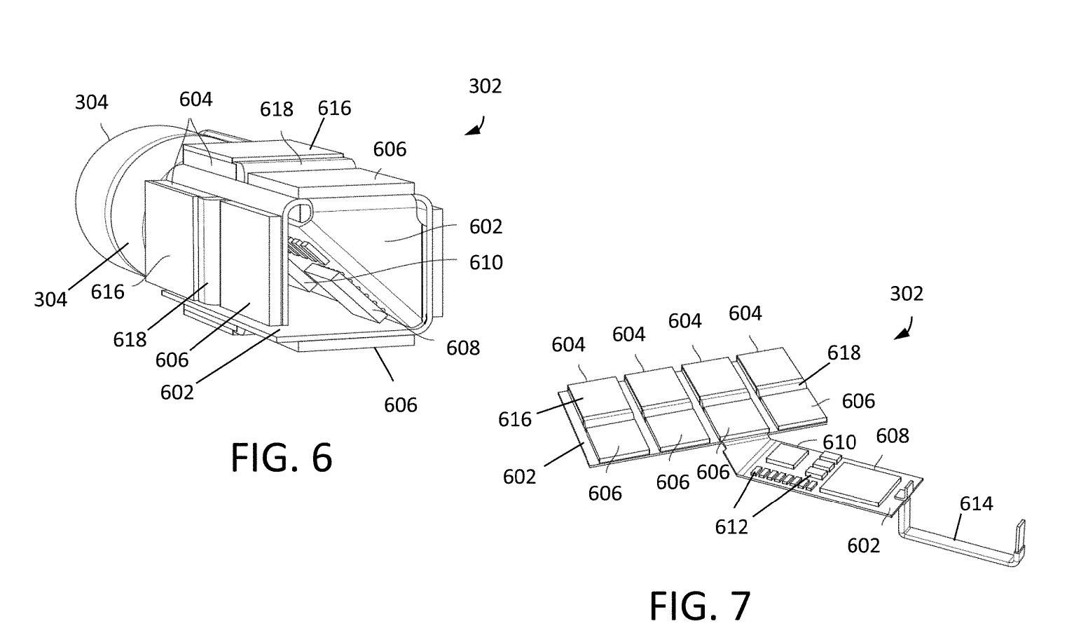

Referring now to FIG. 6 and FIG. 7, an exemplary embodiment of the electronic circuit assembly 302 is depicted in further detail. In particular, FIG. 6 shows additional details of the electronic circuit assembly 302 in a “folded” configuration, similar to the view in FIG. 5, while FIG. 7 shows additional details of the electronic circuit assembly 302 in an “unfolded” configuration (without the batteries 304). As is shown, various electronic components of the electronic circuit assembly 302 are formed on a flex circuit substrate 602 and include, for example: one or multiple ultrasonic transducer (e.g., CMUT) arrays 604, one or more image reconstruction chips 606, a field programmable gate array (FPGA) 608, communications circuitry 610 (e.g., Bluetooth, Bluetooth Low Energy (BLE) chip), discrete circuit elements and/or other devices or sensors 612 (e.g., memory chips, capacitors, resistors, one or more accelerometers/gyroscopes, etc.) and one or more batteries 304 secured by bracket 614. One non-limiting example of a suitable battery type to provide power to the electronic circuit assembly 302 is a zinc air cell, type PR48, size A13. This type of cell may operate at a nominal voltage of about 1.4V with a capacity of about 300 mAh. Other battery types are also contemplated, however.

In particular, sensor devices such as accelerometers, compasses and gyroscopes may provide information to form a position vector over a time series. Additional information may also be determined from such positional information by calculating numerical derivatives (e.g., the derivative of the position is the velocity and the derivative of the velocity is the acceleration). An accelerometer/gyroscope features 3 orthogonal axes (X,Y,Z) that track the position vectors and digitize them at specified intervals. By analyzing these vectors, estimations of the rotation (roll, pitch, yaw) and translation (x,y,z) may be obtained via suitable digital computations using, for example, digital circuitry or a commercial integrated motion processor.

Positional data generated from accelerometers, gyroscopes or other sensors may be handled using any suitable format for digital or analog transfer. In one embodiment, an I²C bus acquires data at regular intervals by a subsystem module. The subsystem module appends a time-stamp with the position data (e.g., 2 bytes of position data from each of 3 accelerometers X, Y, and Z). This data is sent via asynchronous packet information over a USB connection. The positional data may also be blocked during an acquisition and accumulate in an onboard buffer. Gyroscope data may be synchronized to the acquisition data by use of the time-stamp in correlation with the logged time of the acquisition. The interpolation of the position is possible on-chip or off-chip if the exact time does not match between gyroscopic data and acquisition data.

Knowledge of the position of the ingestible ultrasound imaging device during an acquisition provides an ability to combine data collected at different times and at different locations. When collecting data, the position data may be somewhat inaccurate for any of a number of reasons. For example, the gyro sensor may be poorly calibrated or perhaps the acceleration values used to calculate position may accumulate error and relative position may drift. It is also possible that the subject being imaged with the device may have moved relative to the device. In these cases, the position data may be primarily used to estimate stitching offset relationships. In addition, the position data itself can be used to solve not only the stitching offset, but also to update the device location. The corrected position may be estimated by performing a cross-correlation of sensor scans, sub-images, or even sub-volumes where the cross-correlation calculation is restricted to a sub-region of positions within a specified tolerance. The largest correlation values indicate an appropriate stitching region. Other metrics of similarity among regions, such as histogram matching or derivative cross-correlations for example, may also be used to solve for the position offset. In one mode, histograms of rows and histograms of columns may be used to get an accurate registration between images.

Combining scans, images, or volumes may require accurate position locations, which may be obtained in several ways. Once an accurate position of the array is found, a reconstruction algorithm may be used where the two or more scans, images, or volumes can be combined for a single reconstruction. One such reconstruction may be a backprojection of sensor data. Another example may be a scan conversion between consecutive scans.

As is known to those skilled in the art, a flex circuit substrate (such as substrate 602) is a technology used for assembling electronic circuits by mounting electronic devices on flexible plastic substrates, such as a polyimide, a colorless organic thermoplastic polymer such as a Kapton™ film, or transparent conductive polyester film for example. It should be appreciated, however, that the presently disclosed embodiments are not limited to such specific examples of substrate materials.

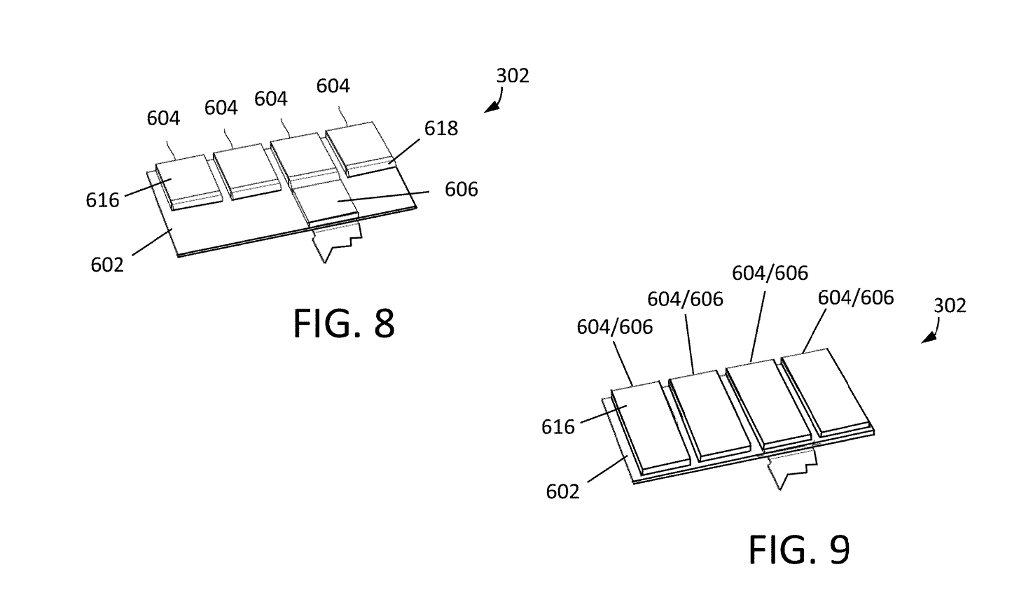

In the specific embodiments depicted, a first portion of the flex circuit substrate 602 of the electronic circuit assembly 302 includes four ultrasonic transducer arrays 604, each having an acoustic protective coating 616 formed thereon. A wire bond encapsulant material 618 (e.g., epoxy) may also be provided to encapsulate and protect any wire bonds (not shown) that may be used to electrically connect an upper portion of the transducer arrays 604 to the flex circuit substrate 602 and/or the associated image reconstruction chip 606. Although FIG. 6 and FIG. 7 depict an arrangement where each transducer array 604 is associated with a corresponding image reconstruction chip 606 disposed adjacent thereto on the flex circuit substrate 602, other arrangements are also contemplated. For example, FIG. 8 depicts an embodiment of the electronic circuit assembly 302 where a single image reconstruction chip 606 serves each of the four transducer arrays 604, and thus in some embodiments two or more of the transducer arrays 604 may share an image reconstruction chip 606. In another embodiment, as shown in FIG. 9, larger area transducer arrays 604 may be used such that they may be monolithically integrated onto a common engineered substrate or a same substrate with the transducer image reconstruction chips 606 in an ultrasound-on-a-chip arrangement. Additional information regarding microfabricated ultrasonic transducers may also be found in U.S. Pat. No. 9,067,779, assigned to the assignee of the present application, the contents of which are incorporated by reference herein in their entirety. Still another possibility is to locate the image reconstruction chips 606 on a different portion of the flex circuit substrate 602.

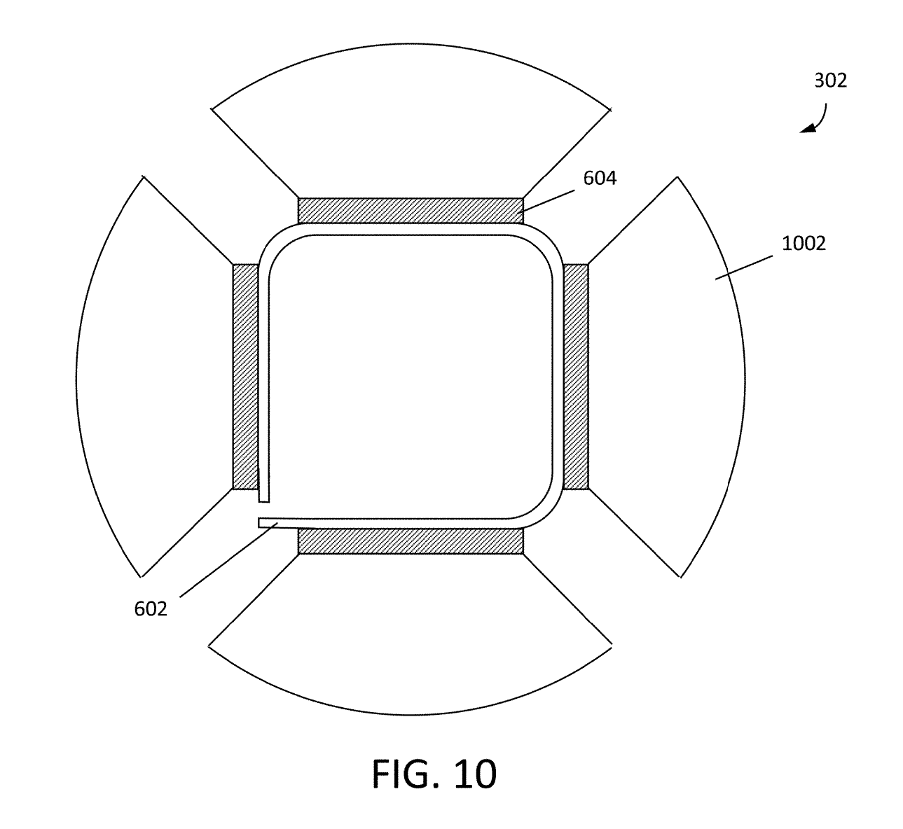

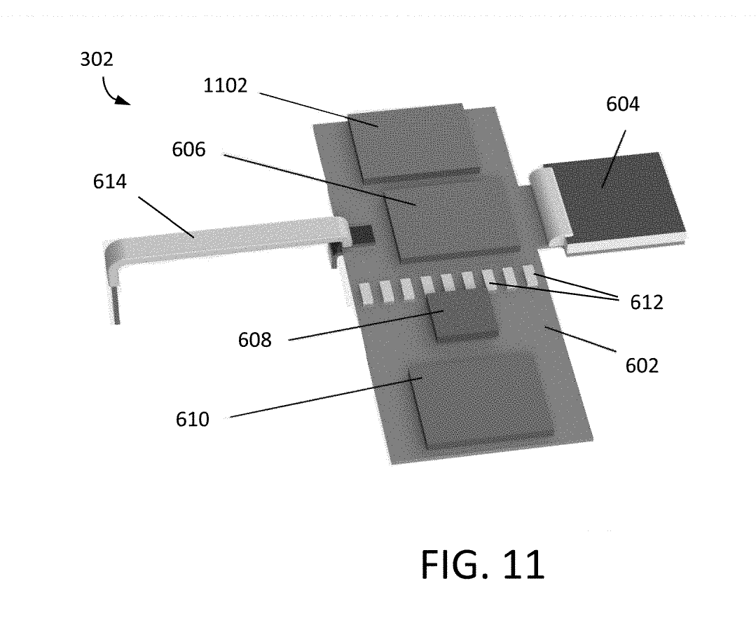

Referring once again to FIG. 6 and FIG. 7, the exemplary embodiment depicted provides an ultrasound imaging device having ultrasonic transducer arrays 604 physically arranged such that the resulting field of view of the device within the pill is equal to or as close to 360 degrees as possible. For example, each of the four transducer arrays 604 may have a field of view of about 90 degrees (45 degrees on each side of a vector normal to the surface of the array) or a field of view in a range of about 40-90 degrees such that the device consequently has a field of view of about 360 degrees or a field of view in a range of about 160-360 degrees. In the non-limiting example of FIG. 6, the flex circuit substrate 602 is fashioned in a generally square shaped arrangement is shaped so as to have a plurality of surfaces having different physical orientations, with each array 604 facing an outward direction about 90 degrees from those on an adjacent surface of the flex circuit substrate. In addition, the portion of the flex circuit substrate 602 on which “non-transducer” components are formed (e.g., FPGA 608, wireless communication chip 610, discrete circuit elements 612, etc.) may be folded and tucked within an interior area defined by the generally square shaped arrangement of the ultrasonic transducer arrays 604. FIG. 10 is a schematic cross-sectional view depicting one exemplary imaging range of view 1002 for the arrangement of arrays 604 in FIG. 6. As can be seen, where each array 604 has a field of view of about 90 degrees, total coverage of about 360 degrees about a longitudinal axis of the electronic circuit assembly may be achieved.

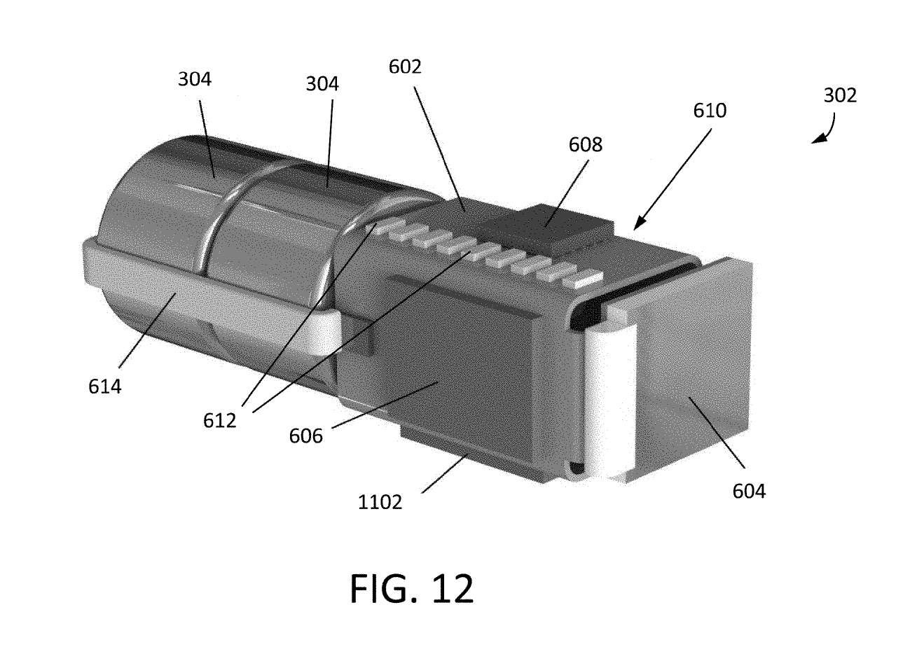

Referring now to FIG. 11, there is shown a perspective view of a single transducer array chip embodiment of the electronic circuit assembly 302. Here, the electronic circuit assembly 302 may include a single transducer array 604 formed on the flex circuit substrate 602. Additional components such as image reconstruction chip 606, FPGA 608, communications circuitry 610, an accelerometer/gyroscope chip 1102, and other discrete components 612 may also be formed on the flex circuit substrate 602. When arranged in a folded configuration as shown in FIG. 12 (with batteries 304 also depicted and communications circuitry 610 on the far side of the folded flex circuit substrate 602), the transducer array 604 may be located in a front facing orientation on an end surface of the folded flex circuit substrate 602, for example in a direction of travel of the pill.

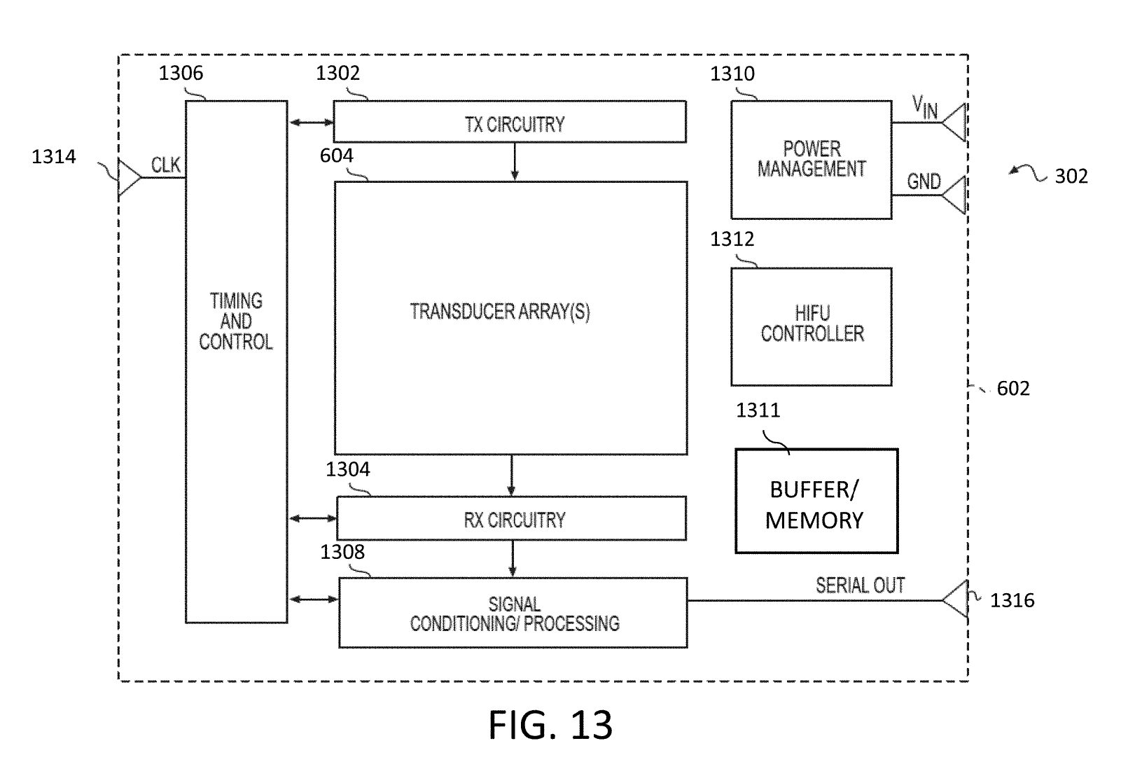

FIG. 13 is a schematic block diagram illustrating at least part of the functionality of the electronic circuit assembly 302 described herein. The various circuits depicted in FIG. 13, while disposed on the flex circuit substrate 602 may reside on multiple chips (e.g., transducer array 604/image reconstruction chip 606) or on a single monolithic ultrasound chip as described above. As shown, the ultrasound imaging device may include the aforementioned one or more transducer arrangements (e.g., arrays) 604, transmit (TX) circuitry 1302, receive (RX) circuitry 1304, a timing and control circuit 1306, a signal conditioning/processing circuit 1308, a power management circuit 1310, a buffer/memory 1311, and optionally a high-intensity focused ultrasound (HIFU) controller 1312. As previously indicated, in one embodiment all of the illustrated elements are formed on a single semiconductor die. In alternative embodiments one or more of the illustrated elements may be instead located off-chip with respect to the transducer arrays 604, such as on one or more image reconstruction chips 606 previously discussed. In addition, although the illustrated example shows both TX circuitry 1302 and RX circuitry 1304, in alternative embodiments only TX circuitry or only RX circuitry may be employed. For example, such embodiments may be employed in a circumstance where one or more transmission-only devices are used to transmit acoustic signals and one or more reception-only devices are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

The one or more transducer arrays 604 may take on any of numerous forms, and aspects of the present disclosure do not necessarily require the use of any particular type or arrangement of transducer cells or transducer elements. Indeed, although the term “array” is used in this description, it should be appreciated that in some embodiments the transducer elements may not be organized in an array and may instead be arranged in some non-array fashion. In various embodiments, each of the transducer elements in the array 604 may, for example, include one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the transducer elements of the transducer array 604 may be formed on the same chip as the electronics of the TX circuitry 1302 and/or RX circuitry 1304 or, alternatively integrated onto the chip having the TX circuitry 1302 and/or RX circuitry 1304. In still other embodiments, the transducer elements of the transducer array 604, the TX circuitry 1302 and/or RX circuitry 1304 may be tiled on multiple chips.

A CUT may include, for example, a cavity formed in a CMOS wafer, with a membrane overlying the cavity, and in some embodiments sealing the cavity. Electrodes may be provided to create a transducer cell from the covered cavity structure. The CMOS wafer may include integrated circuitry to which the transducer cell may be connected. The transducer cell and CMOS wafer may be monolithically integrated, thus forming an integrated ultrasonic transducer cell and integrated circuit on a single substrate (the CMOS wafer). Again, additional information regarding microfabricated ultrasonic transducers may also be found in the aforementioned U.S. Pat. No. 9,067,779, assigned to the assignee of the present application, the contents of which are incorporated by reference herein in their entirety.

The TX circuitry 1302 may, for example, generate pulses that drive the individual elements of, or one or more groups of elements within, the transducer array(s) 604 so as to generate acoustic signals to be used for imaging. The RX circuitry 1304, on the other hand, may receive and process electronic signals generated by the individual elements of the transducer array(s) 604 when acoustic signals impinge upon such elements.

In some embodiments, the timing and control circuit 1306 may be responsible for generating all timing and control signals that are used to synchronize and coordinate the operation of the other elements in the device. In the example shown, the timing and control circuit 1306 is driven by a single clock signal CLK supplied to an input port 1314. The clock signal CLK may be, for example, a high-frequency clock used to drive one or more of the on-chip circuit components. In some embodiments, the clock signal CLK may be, for example, a 1.5625 GHz or 2.5 GHz clock used to drive a high-speed serial output device (not shown in FIG. 13) in the signal conditioning/processing circuit 1308, or a 20 Mhz, 40 MHz, 100 MHz or 200 MHz clock used to drive other digital components on the flex circuit 602, and the timing and control circuit 1306 may divide or multiply the clock CLK, as necessary, to drive other components on the flex circuit 602. In other embodiments, two or more clocks of different frequencies (such as those referenced above) may be separately supplied to the timing and control circuit 1306 from an off-chip source.

The power management circuit 1310 may be, for example, responsible for converting one or more input voltages V_{IN} from an off-chip source (e.g., a battery) into voltages needed to carry out operation of the chip, and for otherwise managing power consumption within the device 100. In some embodiments, for example, a single voltage (e.g., 1.5 V, 5V, 12V, 80V, 100V, 120V, etc.) may be supplied to the chip and the power management circuit 1310 may step that voltage up or down, as necessary, using a charge pump circuit or via some other DC-to-DC voltage conversion mechanism. In other embodiments, multiple different voltages may be supplied separately to the power management circuit 1310 for processing and/or distribution to the other on-chip components.

The buffer/memory 1311 may buffer and/or store digitized image data on the device. In addition to providing capability of retrieving images without wireless connection, the buffer/memory 1311 may also, in the case of a wireless connection, provide support for conditions such as lossy channels, intermittent connectivity, and lower data rates, for example. It will be appreciated that, in addition to storing digitized image data, the buffer memory 1311 may also store control parameters such as those used by the timing and control circuit 1306, for example.

As further shown in FIG. 13, in some embodiments, a HIFU controller 1312 may be included in the electronic circuit assembly 302 so as to enable the generation of HIFU signals via one or more elements of the transducer array(s) 604. It should be appreciated, however, that some embodiments may not have any HIFU capabilities and thus may not include a HIFU controller 1312. Moreover, it should be appreciated that the HIFU controller 1312 may not represent distinct circuitry in those embodiments providing HIFU functionality. For example, in some embodiments, the remaining circuitry of FIG. 13 (other than the HIFU controller 1312) may be suitable to provide ultrasound imaging functionality and/or HIFU, i.e., in some embodiments the same shared circuitry may be operated as an imaging system and/or for HIFU. Whether or not imaging or HIFU functionality is exhibited may depend on the power provided to the system. HIFU typically operates at higher powers than ultrasound imaging. Thus, providing the system a first power level (or voltage level) appropriate for imaging applications may cause the system to operate as an imaging system, whereas providing a higher power level (or voltage level) may cause the system to operate for HIFU. Such power management may be provided by off-chip control circuitry in some embodiments.

In addition to using different power levels, imaging and HIFU applications may utilize different waveforms. Thus, waveform generation circuitry may be used to provide suitable waveforms for operating the system as either an imaging system or a HIFU system. In some embodiments, the system may operate as both an imaging system and a HIFU system (e.g., capable of providing image-guided HIFU). In some embodiments, the same on-chip circuitry may be utilized to provide both functions, with suitable timing sequences used to control the operation within and/or between the two modalities.

In the example shown, one or more output ports 1316 may output a high-speed serial data stream generated by one or more components of the signal conditioning/processing circuit 1308. Such data streams may be, for example, generated by one or more USB 2.0, 3.0 and 3.1 modules, and/or one or more 10 GB/s, 40 GB/s, or 100 GB/s Ethernet modules, integrated on the flex circuit 602. In some embodiments, the signal stream produced on output port 1316 may be routed to wireless communication chip 610 for wireless transmission to a computer, tablet, or smartphone (e.g., computer 106, mobile phone 108 in FIG. 1) for the generation and/or display of 2-dimensional, 3-dimensional, and/or tomographic images. In embodiments in which image formation capabilities are incorporated in the signal conditioning/processing circuit 1308, even relatively low-power devices, such as smartphones or tablets which have only a limited amount of processing power and memory available for application execution, can display images using only a serial data stream from the output port 1316. As noted above, the use of on-chip analog-to-digital conversion and a high-speed serial data link to offload a digital data stream is one of the features that helps facilitate an “ultrasound on a chip” solution according to some embodiments of the technology described herein.

Devices such as that shown in FIG. 13 may be used in any of a number of imaging and/or treatment (e.g., HIFU) applications, and the particular examples discussed herein should not be viewed as limiting. In one illustrative implementation, for example, an imaging device including an N×M planar or substantially planar array of CMUT elements may itself be used to acquire an ultrasonic image of a subject, e.g., a person's abdomen, by energizing some or all of the elements in the array(s) 604 (either together or individually) during one or more transmit phases, and receiving and processing signals generated by some or all of the elements in the array(s) 604 during one or more receive phases, such that during each receive phase the CMUT elements sense acoustic signals reflected by the subject. In other implementations, some of the elements in the array(s) 604 may be used only to transmit acoustic signals and other elements in the same array(s) 604 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device or on a single die.

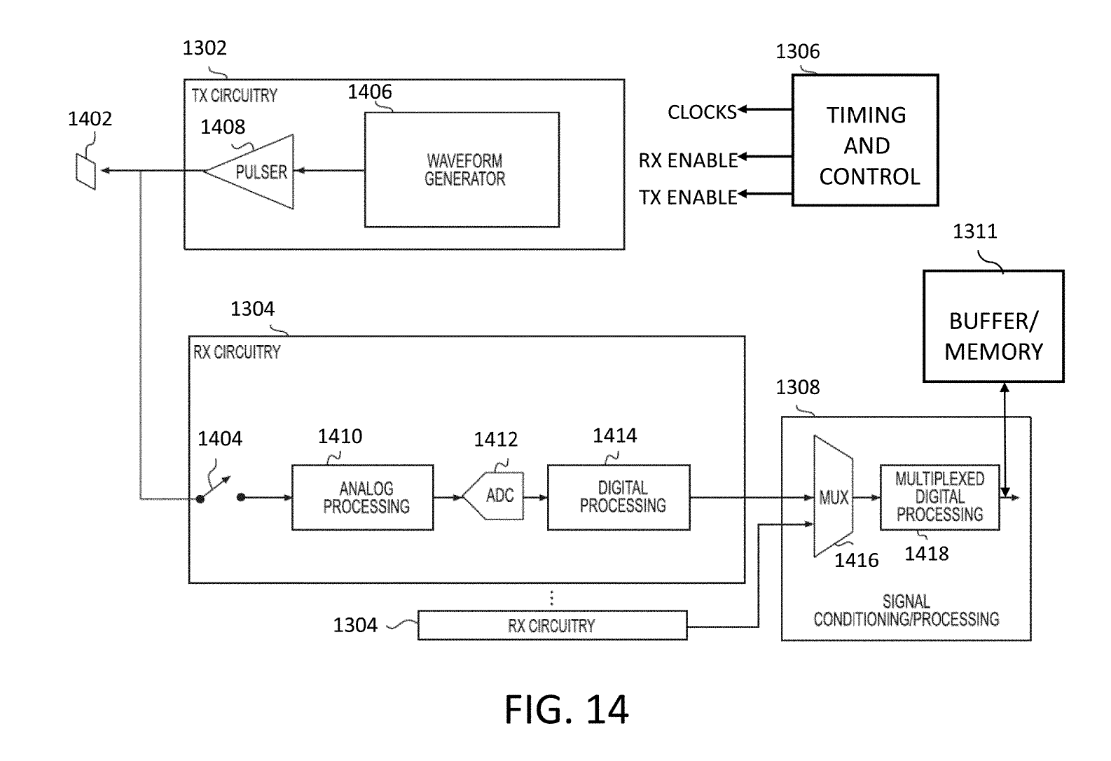

FIG. 14 is a block diagram illustrating how, in some embodiments, the TX circuitry 1302 and the RX circuitry 1304 for a given transducer element 1402 may be used either to energize the transducer element 1402 to emit an ultrasonic pulse, or to receive and process a signal from the transducer element 1402 representing an ultrasonic pulse sensed by it. In some implementations, the TX circuitry 1302 may be used during a “transmission” phase, and the RX circuitry 1304 may be used during a “reception” phase that is non-overlapping with the transmission phase. As noted above, in some embodiments, a device may alternatively employ only TX circuitry 1302 or only RX circuitry 1304, and aspects of the present technology do not necessarily require the presence of both such types of circuitry. In various embodiments, TX circuitry 1302 and/or RX circuitry 1304 may include a TX circuit and/or an RX circuit associated with a single transducer cell (e.g., a CUT or CMUT), a group of two or more transducer cells within a single transducer element 1402, a single transducer element 1402 comprising a group of transducer cells, a group of two or more transducer elements 1402 within an array 604, or an entire array 604 of transducer elements 1402.

In the example shown in FIG. 14, the TX circuitry 1302/RX circuitry 1304 includes a separate TX circuit and a separate RX circuit for each transducer element 1402 in the array(s) 604, but there is only one instance of each of the timing and control circuit 1306 and the signal conditioning/processing circuit 1308. Accordingly, in such an implementation, the timing and control circuit 1306 may be responsible for synchronizing and coordinating the operation of all of the TX circuitry 1302/RX circuitry 1402 combinations, and the signal conditioning/processing circuit 1308 may be responsible for handling inputs from all of the RX circuitry 1304. In other embodiments, timing and control circuit 1306 may be replicated for each transducer element 1402 or for a group of transducer elements 1402.

As also shown in FIG. 14, in addition to generating and/or distributing clock signals to drive the various digital components in the device, the timing and control circuit 1306 may output either an “TX enable” signal to enable the operation of each TX circuit of the TX circuitry 1302, or an “RX enable” signal to enable operation of each RX circuit of the RX circuitry 1304. In the example shown, a switch 1404 in the RX circuitry 1304 may always be opened during the TX circuitry 1302 is enabled, so as to prevent an output of the TX circuitry 1302 from driving the RX circuitry 1304. The switch 1404 may be closed when operation of the RX circuitry 1304 is enabled, so as to allow the RX circuitry 1304 to receive and process a signal generated by the transducer element 1402.

As shown, the TX circuitry 1302 for a respective transducer element 1402 may include both a waveform generator 1406 and a pulser 1408. The waveform generator 1406 may, for example, be responsible for generating a waveform that is to be applied to the pulser 1408, so as to cause the pulser 1408 to output a driving signal to the transducer element 1402 corresponding to the generated waveform.

In the example shown in FIG. 14, the RX circuitry 1304 for a respective transducer element 1402 includes an analog processing block 1410, an analog-to-digital converter (ADC) 1412, and a digital processing block 1414. The ADC 1412 may, for example, comprise an 8-bit, 10-bit, 12-bit or 14-bit, and 5 MHz, 20 MHz, 25 MHz, 40 MHz, 50 MHz, or 80 MHz ADC. The ADC timing may be adjusted to run at sample rates corresponding to the mode based needs of the application frequencies. For example, a 1.5 MHz acoustic signal may be detected with a setting of 20 MHz. The choice of a higher vs. lower ADC rate provides a balance between sensitivity and power vs. lower data rates and reduced power, respectively. Therefore, lower ADC rates facilitate faster pulse repetition frequencies, increasing the acquisition rate in a specific mode.

After undergoing processing in the digital processing block 1414, the outputs of all of the RX circuits (the number of which, in this example, is equal to the number of transducer elements 1402 on the chip) are fed to a multiplexer (MUX) 1416 in the signal conditioning/processing circuit 1308. In other embodiments, the number of transducer elements is larger than the number of RX circuits, and several transducer elements provide signals to a single RX circuit. The MUX 1416 multiplexes the digital data from the RX circuits, and the output of the MUX 1416 is fed to a multiplexed digital processing block 1418 in the signal conditioning/processing circuit 1418, for final processing before the data is buffered/stored in buffer/memory 1311, and/or output from the one or more high-speed serial output ports 1316. The MUX 1416 is optional, and in some embodiments parallel signal processing is performed. A high-speed serial data port may be provided at any interface between or within blocks, any interface between chips and/or any interface to a host. Various components in the analog processing block 1410 and/or the digital processing block 1414 may reduce the amount of data that needs to be output from the signal conditioning/processing circuit 1418 via a high-speed serial data link or otherwise. In some embodiments, for example, one or more components in the analog processing block 1410 and/or the digital processing block 1414 may thus serve to allow the RX circuitry 1304 to receive transmitted and/or scattered ultrasound pressure waves with an improved signal-to-noise ratio (SNR) and in a manner compatible with a diversity of waveforms. The inclusion of such elements may thus further facilitate and/or enhance the disclosed “ultrasound-on-a-chip” solution in some embodiments.

Although particular components that may optionally be included in the analog processing block 1410 are described below, it should be appreciated that digital counterparts to such analog components may additionally or alternatively be employed in the digital processing block 1414. The converse is also true. That is, although particular components that may optionally be included in the digital processing block 1414 are described below, it should be appreciated that analog counterparts to such digital components may additionally or alternatively be employed in the analog processing block 1410.

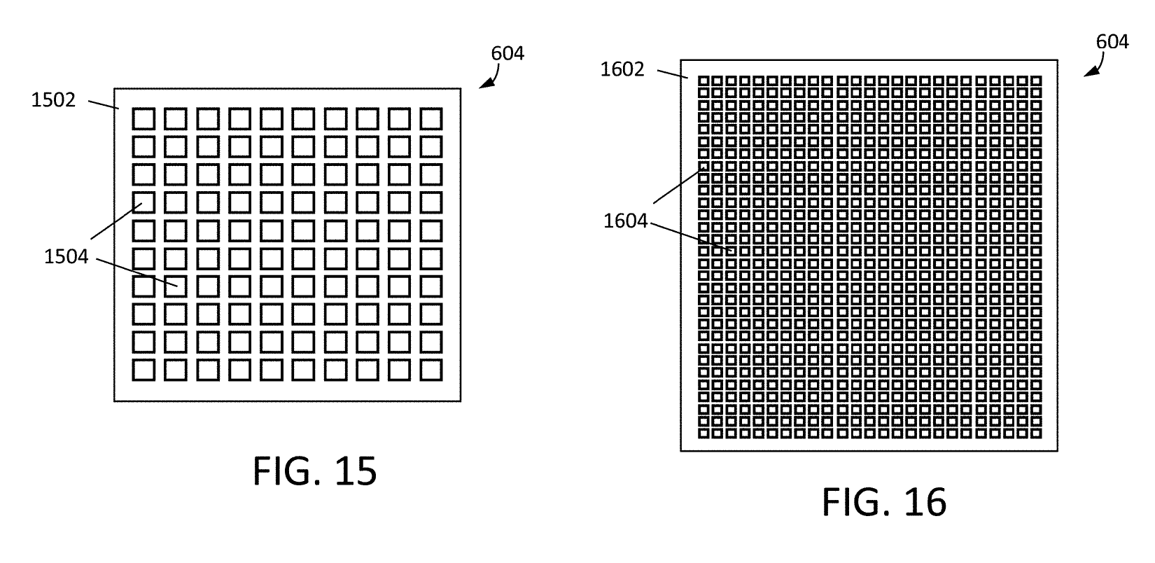

FIG. 15 shows a substrate 1502 of an ultrasound device (e.g., array 604) having multiple ultrasonic transducers (or transducer cells) 1504 formed thereon. In the illustrated embodiment, substrate 1502 includes 100 transducer cells 1504 arranged as an array having 10 rows and 10 columns. However, it should be appreciated that a single substrate ultrasound device may include any suitable number of individual transducer cells having any suitable number of rows and columns or in any other suitable way. In addition, the transducer cells may include shapes such as circular, oval, square or other polygons, for example. Depending on the size of an individual transducer cell and the available area of the substrate, a different number of individual transducer cells may be provided. For example, FIG. 16 illustrates another embodiment of substrate 1602 includes 750 transducer cells 1604 arranged as an array having 30 rows and 25 columns. Other array sizes and configurations are also contemplated, however.

The techniques described herein are exemplary, and should not be construed as implying any particular limitation on the present disclosure. It should be understood that various alternatives, combinations and modifications could be devised by those skilled in the art from the present disclosure. For example, steps associated with the processes described herein can be performed in any order, unless otherwise specified or dictated by the steps themselves. The present disclosure is intended to embrace all such alternatives, modifications and variances that fall within the scope of the appended claims.

What is claimed is:

1. An ultrasound device, comprising:
an electronic circuit assembly, including a plurality of ultrasonic transducers and control circuitry configured to control the plurality of ultrasonic transducers to generate and/or detect ultrasound signals, wherein the plurality of ultrasonic transducers and the control circuitry are integrated on a same substrate; and
an ingestible encapsulating medium that encapsulates the electronic circuit assembly.

2. The device of claim 1, wherein the ingestible encapsulating medium comprises a material that is acoustically conductive.

3. The device of claim 2, wherein the ingestible encapsulating medium comprises a biocompatible material.

4. The device of claim 3, wherein the ingestible encapsulating medium comprises an outer housing into which the electronic circuit assembly is removably inserted.

5. The device of claim 3, wherein the ingestible encapsulating medium comprises a mold.

6. The device of claim 5, wherein the ingestible encapsulating medium comprises a silicone based material.

7. The device of claim 1, wherein the electronic circuit assembly further comprises wireless communication circuitry configured to enable wireless communication between the control circuitry and a host device.

8. The device of claim 7, wherein the host device comprises one or more of a computer, a tablet, and a smartphone.

9. The device of claim 7, wherein the electronic circuit assembly comprises a flexible substrate, on which the plurality of ultrasonic transducers, the control circuitry and the wireless communication circuitry are mounted.

10. The device of claim 9, wherein the flexible substrate is shaped so as to have a plurality of surfaces having different physical orientations, and wherein each of the plurality of surfaces has an individual ultrasonic transducer array mounted thereon.

11. The device of claim 10, wherein adjacent transducer arrays are disposed on surfaces orthogonal to one another.

12. The device of claim 11, wherein each of the adjacent transducer arrays has a field of view in a range of about 40-90 degrees such that device has a total field of view in a range of about 160-360 degrees.

13. The device of claim 10, wherein the flexible substrate comprises a first portion including the plurality of surfaces having an individual ultrasonic transducer array mounted thereon, and a second portion having one or more of: the wireless circuitry, a gyroscope device, an accelerometer device, a compass device, or discrete circuit components formed thereon.

14. The device of claim 13, wherein the second portion is disposed within an interior area defined by a generally square shaped arrangement of the plurality of surfaces of the first portion.

15. The device of claim 13, wherein at least one of the gyroscope device, the accelerometer device, or the compass device is configured to operate with at least another one of the gyroscope device, the accelerometer device, or the compass device to determine device location.

16. The device of claim 15, wherein a first data collection from at least one of the gyroscope device, the accelerometer device, or the compass device at a first time is configured for correlation with a second data collection from at least one of the gyroscope device, the accelerometer device, or the compass device at a second time to determine a relative change in device position.

17. The device of claim 16, wherein the relative change in device position comprises one or more of translation or rotation.

18. The device of claim 9, wherein the flexible substrate is shaped so as to have a plurality of surfaces having different physical orientations, and wherein one of the plurality of surfaces is an end surface having a single ultrasonic transducer array mounted thereon.

19. The device of claim 18, further comprising an acoustic protective coating formed on each individual ultrasonic transducer array.

20. The ultrasound device of claim 1, wherein the plurality of ultrasonic transducers includes a plurality of CMOS ultrasonic transducers.

21. The ultrasound device of claim 1, wherein the plurality of ultrasonic transducers includes a plurality of micromachined ultrasonic transducers.

22. The ultrasound device of claim 21, wherein the plurality of micromachined ultrasonic transducers includes a plurality of capacitive micromachined ultrasonic transducers.

23. The ultrasound device of claim 21, wherein the plurality of micromachined ultrasonic transducers includes a plurality of piezoelectric ultrasonic transducers.

24. An ultrasound device, comprising:
an electronic circuit assembly, including a plurality of ultrasonic transducers and control circuitry configured to control the plurality of ultrasonic transducers to generate and/or detect ultrasound signals;
wherein at least a portion of the control circuitry is integrated on a same substrate with at least one ultrasonic transducer of the plurality of ultrasonic transducers; and
an ingestible encapsulating medium that encapsulates the electronic circuit assembly.

25. An ultrasound device, comprising:
an electronic circuit assembly, including a plurality of ultrasonic transducers and control circuitry configured to control the plurality of ultrasonic transducers to generate and/or detect ultrasound signals, wherein the control circuitry comprises a timing and control circuit, a transmit circuit, and a receive circuit, the timing and control circuit configured to synchronize and coordinate the operation of the transmit circuit and the receive circuit; and
an ingestible encapsulating medium that encapsulates the electronic circuit assembly.

26. The device of claim 25, where in the transmit circuit further comprises a waveform generator configured to generate a waveform that is converted to a driving signal applied to one or more of the plurality of ultrasonic transducers.

27. The device of claim 25, wherein the receive circuit further comprises an analog processing block, an analog-to-digital converter (ADC), and a digital processing block.

28. An ultrasound device, comprising:
an electronic circuit assembly, including a plurality of ultrasonic transducers and control circuitry configured to control the plurality of ultrasonic transducers to generate and/or detect ultrasound signals, wherein the control circuitry comprises a memory device configured to store and/or buffer ultrasound image data therein; and
an ingestible encapsulating medium that encapsulates the electronic circuit assembly.

29. The device of claim 28, wherein the ultrasound image data comprises digitized data.

30. An ultrasound device, comprising:
an electronic circuit assembly, including a plurality of ultrasonic transducers and control circuitry configured to control the plurality of ultrasonic transducers to generate and/or detect ultrasound signals, wherein the control circuitry comprises a power management circuit configured to manage power consumption within the device; and
an ingestible encapsulating medium that encapsulates the electronic circuit assembly.

31. The device of claim 30, wherein the power management circuit is configured to convert one or more input voltages into one or more output voltages for distribution to other components of the control circuitry.

32. A method of performing ultrasound imaging, the method comprising:
receiving, at a host device, ultrasound image data transmitted by an ultrasound device disposed internally within a subject, the host device located externally with respect to the subject;
wherein the ultrasound device comprises an electronic circuit assembly, including a plurality of ultrasonic transducers and control circuitry configured to control the plurality of ultrasonic transducers to generate and/or detect ultrasound signals, wherein the plurality of ultrasonic transducers and the control circuitry are integrated on a same substrate, and an ingestible encapsulating medium that encapsulates the electronic circuit assembly.
